(19) United States
(12) Patent Application Publication
(10) Pub. No.: US 2001/0048733 A1
(43) Pub. Date: Dec. 6, 2001

(54) Method and computer program for checking an image reproduction apparatus, and method for operating a medical apparatus

(76) Inventor: Ulrich Schulze-Ganzlin, Lorsch (DE)

Correspondence Address: SCHIFF HARDIN & WAITE, 6600 SEARS TOWER, 233 S WACKER DR, CHICAGO, IL, 60606-6473, US

(21) Appl. No.: 09/886,072

(22) Filed: Jun. 20, 2001

(30) Foreign Application Priority Data
Jun. 21, 2000 (DE) 100 290526.6

(51) Int. Cl. G01N 23/04; G06K 9/00; A61B 5/05

(52) U.S. Cl. 378/62; 382/130; 600/425

(57) ABSTRACT

In a method and computer program for checking an image reproduction device a test zone is included as a partial region of the displayed image. An image parameter is assigned to the test zone and a partial region of the test zone is altered with regard to the image parameter. An input by the user is detected upon identification of a difference in the image parameter between the partial region and the remaining test zone. The image parameter of the partial region is stored and it, or of a transformation thereof, is composed with a predetermined guide value for the image parameter.

### BACKGROUND OF THE INVENTION

1. Field of the Invention

The present invention relates to a method for checking an image reproduction apparatus, in particular a medical diagnostic apparatus, and a computer program therefor, and a method for operating a medical apparatus.

2. Description of the Prior Art

In medical technology in which images are displayed and used for diagnosis, the quality of the image-reproducing apparatuses and the ambient conditions thereof play an important part.

The image reproduction apparatus is part of equipment for carrying out computed tomography imaging, ultrasound imaging and X-ray imaging such as, for example, mammographic imaging or dental panoramic recordings, which have a digital image stored in a memory. This digital image has to be made available for observation by the user, for which purpose it is displayed on an image reproduction apparatus. In this case, the quality of the display of the digital image on the image reproduction apparatus critically depends not only on the ambient illumination, but also on the specific settings of the image reproduction apparatus itself. Furthermore, the generation of the image signal from the digital image data, stored as pixel values, by means of a graphics card and the transformation characteristic for the assignment of pixel values into color values, also influences the quality of the display.

In practice, light intensity measuring methods are employed in which, using a sensor, the light intensity of the switched-off image reproduction apparatus and the light intensity of a brightest point of the image reproduction apparatus are measured and, using the measured values, the image reproduction apparatus is set with regard to brightness and contrast. This setting must not be altered again and the ambient conditions which were present during the measurement basically have to be maintained throughout the operation of the image reproduction apparatus for diagnostic purposes. As a rule, it is necessary to correspondingly darken the room in which the image reproduction apparatus is installed.

### SUMMARY OF THE INVENTION

The method according to the invention enables checking of image reproduction apparatuses, by arranging a test zone as a partial region of the displayed image, measuring an image parameter such as contrast, resolution, noise of the test zone, altering a partial region of the test zone with regard to the image parameter, detecting an input by the user upon identification of a difference in the image parameter between the partial region and the remaining test zone, storing the image parameter of the partial region and, finally, comparing the image parameter or of a transformation thereof with a predetermined guide value for the image parameter.

By comparing the image parameter of the partial region with the image parameter of the remaining test zone, for example a surrounding region, it is possible to derive a characteristic quantity which allows a conclusion as to whether the quality of the image reproduction corresponds to the diagnostic requirements. As a result, it is also possible to ascertain altered ambient conditions and even changed settings on the image reproduction apparatus and to check them in respect of the suitability of the reproduction apparatus for medical diagnosis. The dynamic alteration of the partial regions of the test zone and the time when alterations are perceived are a measure of the reproduction quality of the system including the present illumination situation and physiological constitution, in particular adaptation of the eyes of the person evaluating the image.

It is understood that the method can also proceed in the opposite direction, specifically when, proceeding from a difference, an input is entered precisely when it is no longer possible to identify a difference.

The test zone is advantageously part of a standardized test pattern, in particular an SMPTE test pattern (Subcommittee of the Society of Motion Picture and Television Engineers).

The use of a standardized test pattern allows the user to identify further properties of the image reproduction apparatus; moreover, it is made clear to the user that the determination of properties of the image reproduction apparatus which affect the quality of the image reproduction is involved.

If an SMPTE test pattern is used, it is appropriate for the low-contrast elements 95% and 5% of the test pattern to be used as test zones and altered dynamically, since these zones already contain altered regions.

At this point, however, it should be noted that the test zones alternatively can be arranged in an image intended for diagnosis it is even possible for the test zones to be superimposed over the actual image data in order to be able to ascertain the corresponding quality of the image reproduction apparatus precisely for this region of interest.

In an embodiment, at least two test zones are interrogated. The test zones are advantageously spaced apart from one another and can be interrogated one after the other. This is appropriate in particular when one test zone is bright and one test zone is dark, in order to prevent saturation of the eye as a result of alternate focusing on a dark region and a bright region.

The test zone in the initial state advantageously lies in the reproduction-specific problem range of the image parameter, so that a measurement already enables a conclusion about the quality of the image reproduction. If two test zones are used, in the initial state they may lie in the respectively opposite end range of the image parameter. It goes without saying that it is generally not necessary to carry out measurements in those ranges of the image parameter in which problems are not normally to be expected.

In order to avoid incorrect inputs by the user upon identification of a difference in the image parameter, the image parameter of the partial region is altered with a time delay. If an input is made during this time delay, this is identified as an error and the method has to be carried out anew.

In order to avoid the risk of arbitrary inputs for shortening the method in a manner contrary to intentions, the time delay is effected in a randomly oriented manner, so that the user does not know when the alteration of the partial region of the test zone with regard to the image parameter actually begins. If confirmation is given before the beginning of the alteration, this is identified as an error and the method has to be carried out anew.

The luminance of the image reproduction apparatus is advantageously detected while the method is being carried out. This has the advantage that the luminance thus detected can be compared with the luminance during the medical diagnosis of the image displayed on the image reproduction apparatus and, in the case of excessively large deviations of the luminance, the risk of mis-diagnosis can be indicated. Furthermore, certain subfunctions of the system may be disabled; for example, it is possible for the display of the image not to take place under these conditions. A high degree of operational reliability is achieved with the additional detection of the luminance.

The partial region and the surrounding region advantageously have image parameters that are homogeneous over their respective areas, so that an area-dependent impression is produced and the image values can be better compared.

The contrast and/or the resolution and/or the noise is advantageously used as the image parameter, a black-and-white image preferably being used. Of course, if color images are employed, it is also possible to use the contrast of other colors.

The method can be used such that, in the region in which specific contrast differences cannot be identified, no or correspondingly fewer contrast values are used for the representation of the digital image. As a result, it is possible to improve the evaluation of digital images even in the case of reduced quality of the image reproduction apparatus, although care must be taken to ensure that a minimum quality is present.

In accordance with a further embodiment of the method for operating medical apparatuses and for influencing the functional sequences thereof, in which a parameter is determined and the functional sequences are controlled as a function of the parameter, the parameter is determined from the determination of the behavior of the reproduction apparatus with regard to an image parameter, taking account of the predetermined boundary conditions.

In this case, the luminance at the time of the determination of the behavior of the reproduction apparatus with regard to an image parameter can be compared with the luminance at the time of the actual utilization of the medical apparatus, and, in the case of deviations going beyond a predetermined tolerance range, the functional sequences can be controlled as a function of the parameter.

The invention further relates to a method for the reproduction of image data on image reproduction apparatuses in medical diagnosis, in which the luminance at the time of checking is compared with the luminance at the time of the actual utilization, the image data being reproduced on the image reproduction apparatus only in the case of deviations which lie within a predetermined tolerance range.

The invention also relates to a computer program for carrying out the above-described method.

### DESCRIPTION OF THE DRAWINGS

FIG. 1 is a computer program flowchart for implementing the inventive method, with respective flowcharts for subroutines shown in FIGS. 1a and 1b.

FIG. 2 illustrates a test pattern with two test zones, respectively shown in detail in FIGS. 2a and 2b, in accordance with principles of the present invention.

FIG. 3 shows an example of measured characteristic curve of an image reproduction apparatus for explaining the inventive method.

FIG. 4 illustrates an apparatus for implementing the inventive method, having a number of sensors for detecting brightness.

FIG. 5 is a flowchart for a method for operating a medical apparatus, which includes the method of FIG. 1.

### DESCRIPTION OF THE PREFERRED EMBODIMENTS

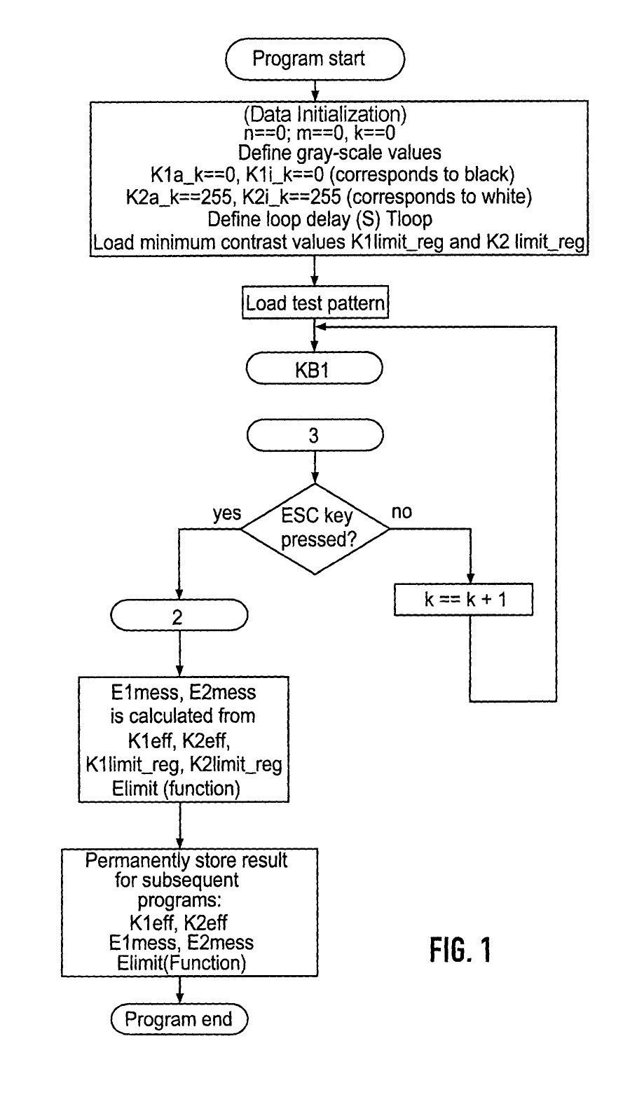

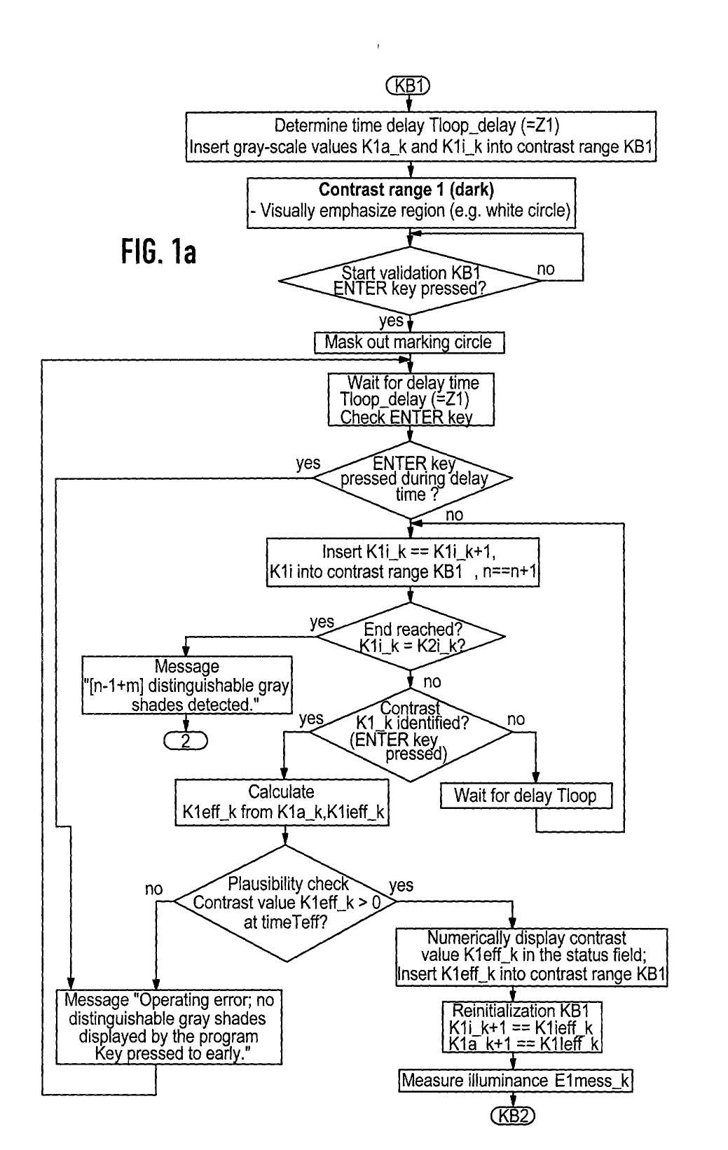

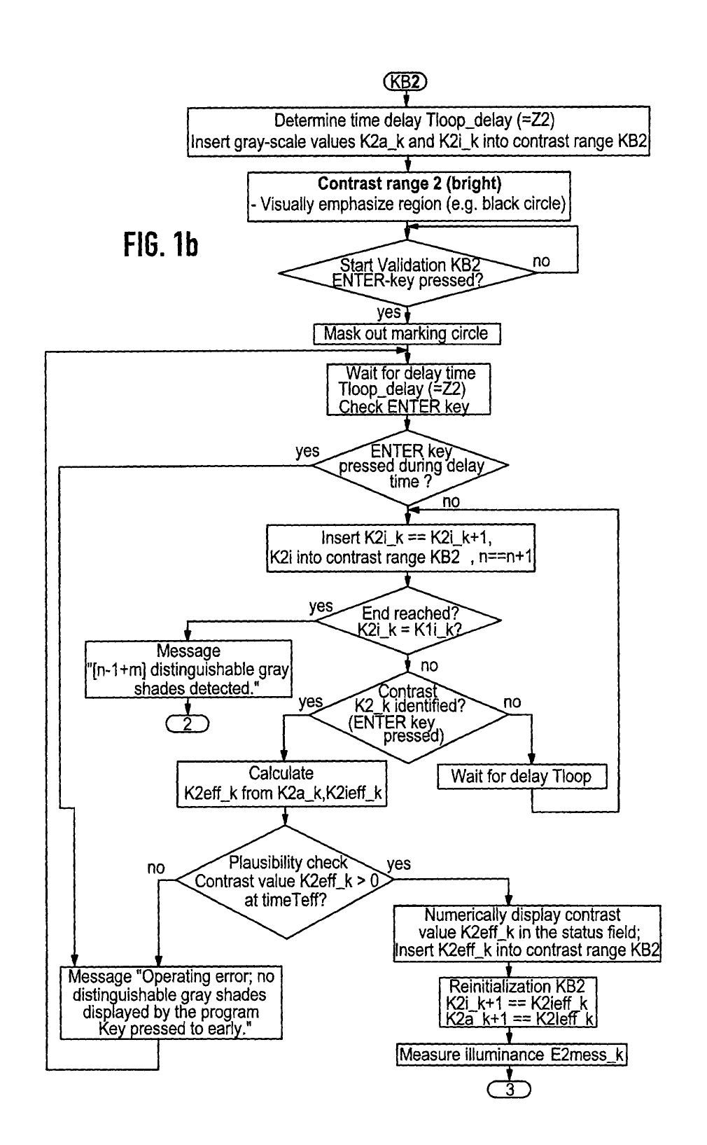

A program flowchart for implementation of the method according to the invention on a computer (not illustrated) is shown in FIGS. 1, 1a and 1b. The starting point for the method is a test pattern stored in a digital memory. In the representation of the test pattern the digital image pixels are converted into analog video signals or into corresponding digital image reproduction signals in the case of digital display apparatuses such as flat screens. Using these video signals or digital image reproduction signals, a representation is effected on the image reproduction apparatus and can be observed there by the user.

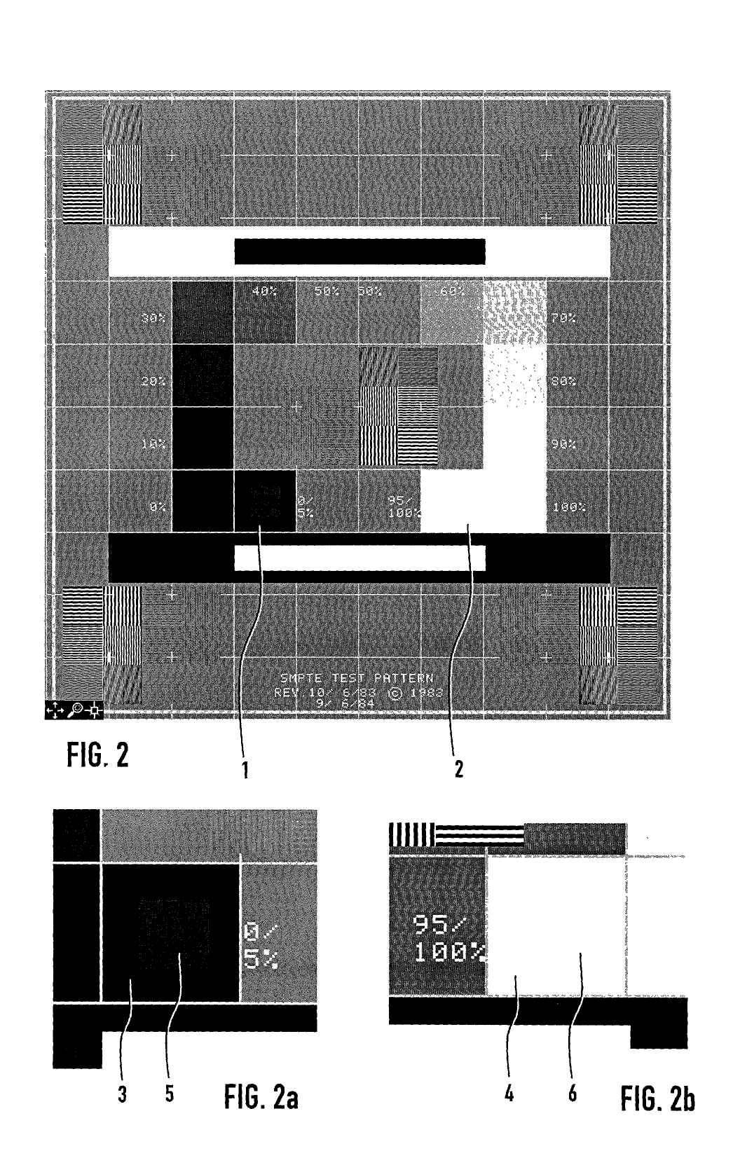

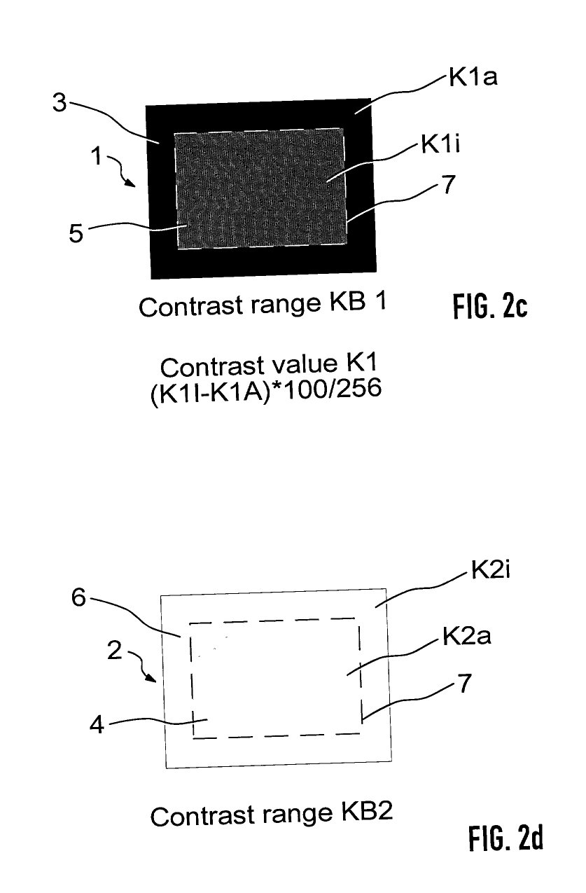

The test pattern, illustrated in FIG. 2, has different contrast ranges, two of which are designed as test zones 1, 2 having variable contrast values. Each of the test zones 1, 2 consists of a surrounding region 3, 4 having a gray-scale value K1a and K2a, respectively, and a partial region 5, 6 embedded completely in the surrounding region 3, 4 and having a gray-scale value K1i and K2i, respectively.

In the case of the test pattern which is illustrated in FIG. 2 and is formed from gray-scale values having a contrast value between 0 and 255, the gray-scale values for the surrounding region 3 and the partial region 5 and also the surrounding region 4 and the partial region 6 are fixed at the beginning of the method. In the test zone 1 the gray-scale values are initially fixed at 0, which corresponds to complete blackening, and in the test zone 2 the gray-scale values are fixed at 255, which corresponds to a white area. Accordingly, the representation of the test zones 1, 2 is initially completely uniform.

When the test pattern is loaded into the computer memory area provided for output of the image, the gray-scale values for the test zones 1 and 2 that have been fixed during data initialization are likewise read in and displayed on the image reproduction apparatus. The partial region 5, 6 of the test zone 1, 2 in which the changes are to be expected is emphasized by a marking 7 in the form of a broken line. Actuation of the enter key starts the alteration of the gray-scale value of the partial region 5. In this case, first the marking is masked out, so that the entire test zone 1 is colored uniformly. After a random delay time Z1 prescribed by the computer, the gray-scale value K1i of the partial region 5 is altered by a gray-scale value, the gray-scale value K1i being increased in this case. Actuation of the enter key communicates identification of the alteration of the contrast value. If the enter key is not pressed, then the gray-scale value is continuously increased until there is a perceptible gradation of the partial region 5 relative to the surrounding region 3. After the enter key has been pressed the contrast value K1 is calculated, stored and inserted in a status field. The contrast value K1 of the test zone 1 is calculated as K1=((K1ieff−K1a) *100/256). For a repeated performance of this method sequence, a reinitialization of the gray-scale value of the test zone 1 is performed in such a way that the identified gray-scale value K1i of the partial region 5 is also assigned to the surrounding region 3 having the contrast value K1a, but without the display already being adapted to the new value at this moment.

If the enter key is pressed before the delay time Z1 has elapsed, an error message appears and the program section or this method step has to be repeated again in its entirety.

Once the first contrast value in the dark range has been ascertained, the test zone 2 with the opposite gray-scale value is then altered, in this case beginning at 255. To that end, the gray-scale values K2a and K2i originating from the data initialization are loaded into the test zone 2 and the partial region 6, whose gray-scale value K2i will be altered, is visually emphasized by a marking 7. In order to initiate the alteration of the gray-scale value in the region 6, the enter key must then be pressed, whereupon the marking 7 is masked out and a delay time Z2 begins. After the delay time has elapsed, the gray-scale value is reduced and there is a wait during a time interval S.

In order to ascertain whether the altered contrast between the partial region 6 and the surrounding region 4 is identified, this must be confirmed by pressing the enter key. If this is not the case, the gray-scale value is reduced until the enter key is pressed. From the data that are then present, the contrast value K2 is calculated, stored and inserted in the status field. The contrast value K2 is calculated as a percentage according to the formula K2=100−ABS(((K1a−K1i)*100/256).

A reinitialization of the gray-scale value K2a is subsequently performed in that the ascertained, identified gray-scale value K2i of the partial region 6 is also assigned to the surrounding region 4 having the gray-scale value K2a.

If the escape key (ESC) is then pressed or the gray-scale values of the partial region 5 or 6 reach the other end of the range, that is to say K1i=255 or K2i=0, then the program run is concluded and the stored results for the contrast values K1i and K2 which were ascertained for each individual sequence can be output on a connected printer in the form of a characteristic curve for the monitor.

An SMPTE test pattern consisting of many different regions is reproduced in FIG. 2. Inter alia, zones having different contrasts of 0, 10, 20, . . . to 100% are provided; furthermore, bar gratings having a different line density are provided in the center and in the corners. Two test zones 1, 2 in the region of the zones of different contrast are of importance here. The test zones 1, 2 are illustrated in detail in FIGS. 2a and 2b and diagrammatically in FIGS. 2c and 2d. The test zone 1 from FIG. 2a consists of a surrounding region 3 in which a partial region 5 is completely embedded, the partial region 5 having a higher contrast than the surrounding region 3, and the gray-scale value of the partial region 5 being higher than the gray-scale value of the surrounding region 3.

FIG. 2b illustrates the test zone 2 in detail, and it can be seen that a partial region 6 is embedded in the surrounding region 4, the contrast values of which partial region 6 are lower than those of the surrounding region 4.

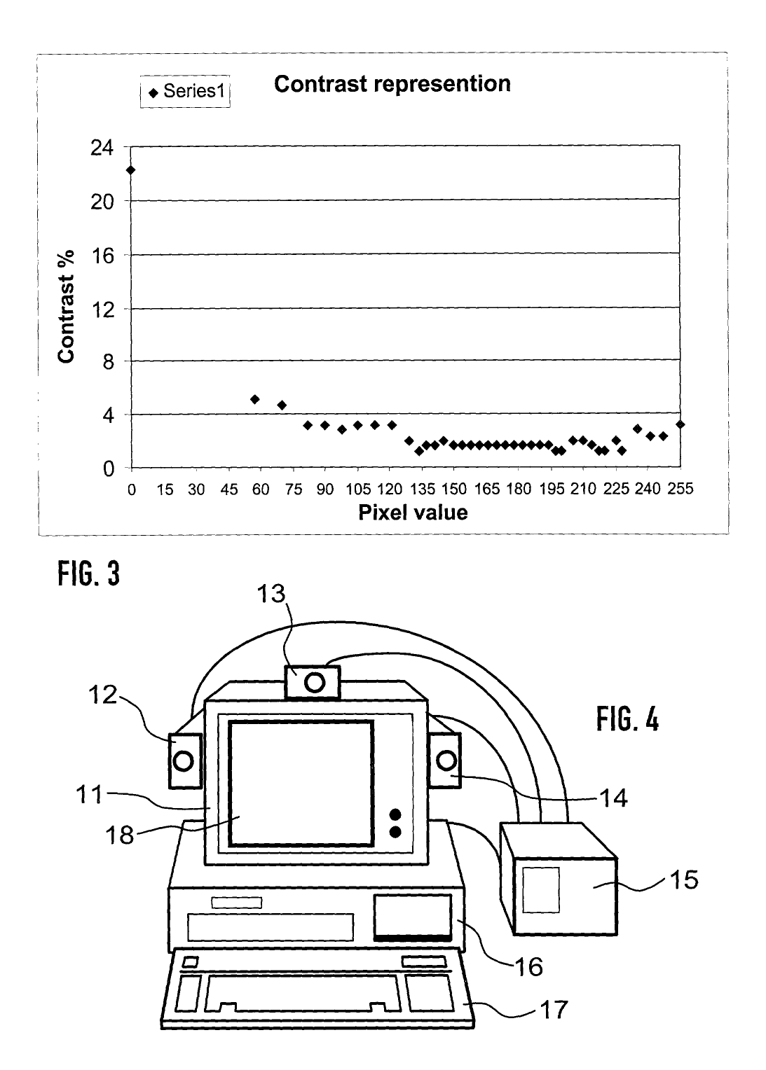

FIG. 3 shows a characteristic curve for an LCD monitor which has been produced through execution of the program shown in FIG. 1. The plotted measured values were formed in such a way that the number of required gray-scale values until ascertainment of the next contrast level was ascertained; in other words, this is the difference between the value K1 a and the value K1i, the ascertained number of gray shades having been divided by the number of gray shades available in total, i.e. 256, and having been multiplied by 100 in order to obtain a percentage value. The first value is formed as follows: in the case of a surrounding region 3 having a contrast value K1a=0, that is to say a black region, the contrast value K1i of the partial region 5 was continuously increased until a perceptible alteration of the contrast relative to the surrounding region was ascertained at a value K1i=57. It can be seen that the gray-scale values—arranged in this range—of the image used for diagnosis are completely unsuitable for reproduction on the image reproduction apparatus since the image reproduction apparatus does not yield any distinguishable image information in this range.

Only the next value, which, after a further 13 gray shades, effects a perceptible change in contrast of the partial region 5 relative to the surrounding region 3, points into a range without further outliers. The contrast value of about 5% for the second value is nevertheless still too high for many applications of X-ray diagnosis, so that a sufficient resolution of the gray-scale values is provided only in the range in which further reduced contrast values of less than 2% are attained.

Upon examination of the characteristic curve from FIG. 3, only 39 gray-scale values are identified instead of 256 possible gray-scale values, in other words the image reproduction apparatus actually offers only 39 distinguishable gray-scale values instead of 256 gray-scale values. Since the digital image data are distributed between 256 gray-scale values, the gray-scale values of the image data must be corrected in such a way that the 256 gray-scale values are converted to 39, in order to avoid an information loss that is as low as possible. With this correction, the range of gray-scale values to be displayed can be enlarged when the digital image data do not exhaust the full range of 256 gray-scale values but rather have for example only 100 different gray-scale values. In this case, the resolution is increased from 256/39 to 150/39. In an advantageous manner, only gray-scale values in the boundary region are left out of consideration, in order not to corrupt the characteristic curve of the image data.

It goes without saying that instead of 256 gray-scale values in an 8-bit representation, it is also possible to use 1024 gray-scale values in a 10-bit representation or any other number of gray-scale values.

FIG. 4 illustrates an apparatus configuration for carrying out the method. It shows sensors 12-14 arranged on an image reproduction apparatus 11 and serving for determining the ambient luminance during the performance of the method and also later during operation of the image reproduction apparatus 11. The sensors 12-14 are connected via a connecting apparatus 15 to a computer 16, to which a keyboard 17 and a screen 18 are also connected. The image present for medical diagnosis is displayed on the screen 18 during operation.

Even if the brightness and/or the contrast on the screen 18 is adjusted by means of regulators, diagnosis errors can be avoided by means of the present method or computer program.

FIG. 1 furthermore shows that, in the program region in which the gray-scale values of the partial region 3, 4 are increased and are to be assessed by the observer, the reaction time is taken into account in that, during inputting, an average reaction time is assumed, and is calculated back to the value which corresponds to the identified value K1eff. If the user thus identifies a difference in contrast, a reaction time elapses until actuation of the entry key, which informs the program that the change in contrast has been identified. During this time, however, the contrast of the partial region is increased further, so that the gray-scale value then ascertained is higher than the value actually already identified. The effectively identified value is subsequently displayed in the test zone.

As an alternative to this procedure, care could also be taken to ensure that, after each change in the gray-scale value, a sufficient time for taking account of the reaction time is dimensioned. As a result, however, the time for implementing the method is significantly prolonged. Assuming a gray-scale value range of 256 gray-scale values, each individual one of which is identified, a waiting time of 1 second after each alteration of the gray-scale value would mean a time requirement of 256 seconds which amounts to more than four minutes. In particular when the alteration of the gray-scale value is not identified, the full length of time elapses, so that occasionally the observer has to focus on the test zone for a number of seconds, which can lead to fatigue. However, the results are more accurate in this case.

As an example, the method according to the invention can be carried out regularly after the monitor is switched on or, alternatively, at any other previously stipulated point in time, for example after the warming-up of the screen, in order to obtain information about the quality of the image reproduction. In this case, it is also possible, in particular, additionally to ascertain the influence of the ambient illumination by means of a sensor in order then to enable a check during medical diagnosis of the image displayed on the image reproduction apparatus.

The brightness itself is not directly ascertained using the method described herein. It may be the case, however, that the gray shades cannot be ascertained when the brightness of the image reproduction apparatus is not high enough, which depends, inter alia, also on the luminance of the surroundings. Without complex measurement of the light density of the image reproduction apparatus, it is possible to ascertain that there is a sufficient contrast for medical diagnosis of images, as, for example, may even be prescribed in an application-specific manner for different areas in varying magnitudes. As an example, the contrast might no longer suffice for mammography, but a dental diagnosis might still be possible.

If, as a result of the method, it is ascertained that the image reproduction apparatus contrast to be identified is inadequate for enabling a medical diagnosis, it is possible, if appropriate, to change the setting of the image reproduction apparatus by turning at the brightness regulator or the property of the image reproduction apparatus at the contrast regulator and, when the method is carried out anew, a quality that is then sufficient can be ascertained.

If specific performance properties of the image reproduction apparatus are not achieved, functions of the medical diagnosis may be restricted, disabled or not used until after confirmation by the user. In this case, in particular, display of images may be refused or entry of findings into the computer may be disabled. If the method is carried out before the creation of the recording which yields the digital image data, then the creation of the recording may be disabled in the event of specific performance properties not being achieved. This avoids the situation whereby work is performed on a faulty system which does not permit adequate medical diagnosis.

In this case, the degree of restriction is oriented to the currently still discernable contrast K1 and K2 determined in accordance with the diagram from FIG. 1, the ambient luminance ETO at the time of measurement, the technology and modality of image acquisition, for example ultrasound, CT, mammography, dental, the medical indication, for example, findings, monitoring, in the dental field root-canal treatment, caries detection, the ambient luminance, the ETN at the time when the reproduction system is used. By virtue of the method, it is possible to determine equidistant contrast values over the entire brightness range. As a result, it is possible to determine characteristic curves for improving the representation quality through compensation of non-ideal representation properties of the reproduction apparatus. Thus, if specific pixel value spacings are not identified, in these regions no or correspondingly fewer pixel values are used for gray-scale value representation. The method thus has the advantage over a light intensity measurement method that the image reproduction chain is taken into account, namely digital value of the test pattern, conversion into video signal by means of the graphics card, the graphics mode, the software, the cables, representation on the image reproduction apparatus with a specific luminance and a characteristic curve under conditions of ambient illumination and the constitution of the observer.

The method can contribute to quality management at two essential points, in that the method results determined can be documented in order to prove the system and method quality used.

The method results used may be the basic data of the quality examination, namely the date of the quality examination of the reproduction apparatus, the data for identification of the reproduction system, the name of the person who carried out the quality examination, the results of the contrast determination—K1eff(k) and K2eff(k) where k≧1, the luminance on the image reproduction apparatus—Emess(k), where k≧1, the number of gray shades determined, the name and the version of the program used, or the permissible luminances in dependence on functions—E1imit (function).

The following data are to be regarded as findings-related data, namely the date of the findings, the data for identification of the reproduction system, the name of the person who made the findings, the results of the contrast determination—K1eff(k) and K2eff(k), where k≧1, the luminance on the monitor—Emess(k), where k≧1 and the name and the version of the program used.

Documentation site may be part of the readable findings text and thus part of the findings, but also in the data record of the findings test or of the found image.

Image parameters include the contrast, the spatial resolution, the noise and combinations thereof.

In the case of contrast, this means that it is possible to use areal, homogeneous structures for examining the minimum contrast representation both in the gray and in the color range. The dynamic element is the insertion of an area whose color or gray-scale value is inserted with continuously increasing contrast with regard to the surrounding area.

Spatial resolutions can be determined using lines arranged next to one another. In this case, the lines differ from one another in a high-contrast manner, e.g. black/white of 0/255. The parameter for resolution could be the number of line pairs per length. The dynamic element then results from the continuous reduction of the line pairs per length.

The noise requires an arrangement similar to that in the case of contrast. The average gray-scale or color value remains constant; the distribution of the values will change.

Also conceivable, moreover, is an arrangement comprising the combination of the previous embodiments, e.g. constant line pair representation but with variable contrast.

As described, as a function of the present settings and the luminance, it is possible to determine an individual characteristic curve of the image reproduction apparatus. If the characteristic curve is known, compensation can be effected by transforming the characteristic curve, so that it specifies substantially equidistant contrast values which are still just identified in the special case just noticeable differences—JND). Ideally, the number of pixel values to be represented in gray shades or color contrast levels should be less than or equal to the JND number.

Each JND(n) may occupy a range n in the reproduction characteristic curve. The pixel values are assigned to these ranges by assignment table. If there are more pixel values than JND, amalgamation has to be effected. Ideally, in order as far as possible not to falsify the image impression through nonlinear mapping, averaged amalgamation is effected.

The method has been described such that values are altered until an alteration becomes visible. The method may also proceed in the opposite direction, specifically when, proceeding from a difference, a difference is no longer discernable.

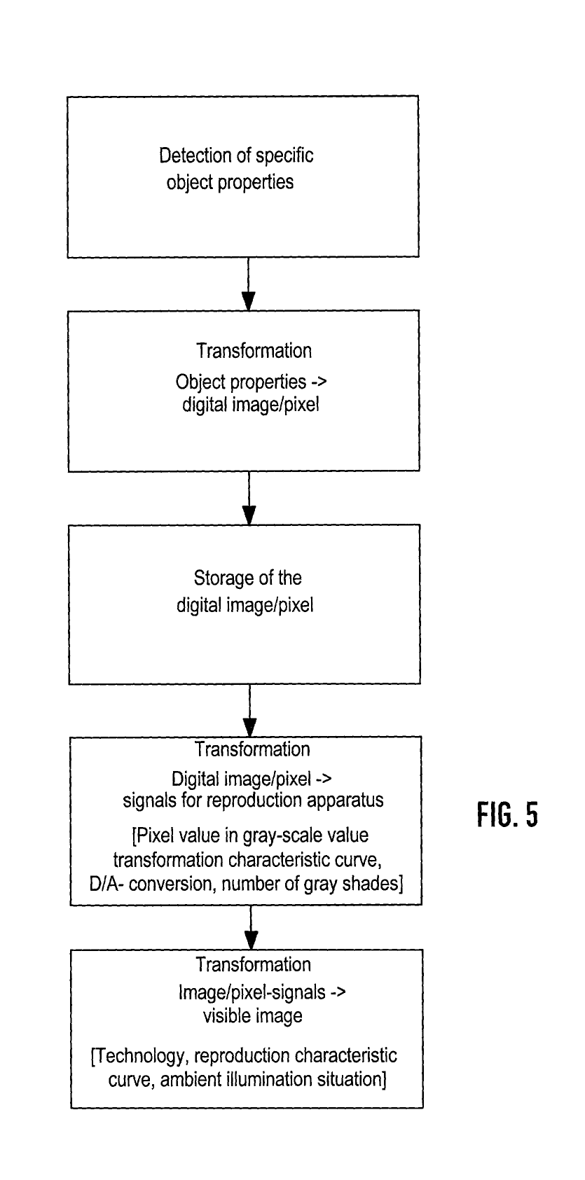

FIG. 5 illustrates a flowchart for the superordinate method in which the method described in FIG. 1 is embedded. First of all, the type of object to be examined is selected and the suitable method, for example X-ray or ultrasound, is chosen. Afterwards, the object properties are detected by a suitable sensor and transformed into a digital image constructed from pixels. The resulting digital image data are stored. In order to prepare for the display of the digital image data, the latter must be transformed into signals for a reproduction apparatus. This is followed by further transformation of the signals into a visible image. In this case, it must be ensured that the quality requirements for representation of the image are complied with in order to allow a sufficiently reliable medical diagnosis.

The following abbreviations are used in the program flowchart in accordance with FIG. 1:

TABLE 1

| | | Typ. range |
|---|---|---|
| Variable | Designation | of values |
| K1a | Gray-scale value in contrast | 0-255 |
| | range KB1-outside | |
| K1i | Gray-scale value in contrast | 0-255 |
| | range KB1-inside | |
| K2a | Gray-scale value in contrast | 0-255 |
| | range KB2-outside | |
| K2i | Gray-scale value in contrast | 0-255 |
| | range KB2-inside | |
| n | n-th incremental loop in KB1 | |
| m | m-th incremental loop in KB2 | |
| k | k-th pass through KB1 and KB2 | |
| K1_k | Determined contrast value from range KB1 | 0.1-10% |
| | at the k-th pass | |
| K2_k | Determined contrast value from range KB2 | 0.1-10% |
| | at the k-th pass | |
| Emess_k | Luminance, measured by luxmeter, on the | 801 x |
| | monitor at the k-th pass | |
| Emess(k) | Vector with Emess_k | |
| E1imit | Vector, calculated from K1_k | 2001 x |
| (function) | K2_k, Emess_k, K1limit_reg and | |
| | K2limit_reg, specifies what | |
| | functions are permitted at what | |
| | max. luminance. | |
| Tenter | Time at which the ENTER key is | |
| | pressed | |
| Teff | Effective time for determining | |
| | contrast value: Tenter minus | |
| | Tuser_delay | |
| K1eff_k | Calculated contrast in the range | |
| | KB1 at the time Teff | |
| K1eff(k) | Vector with K1eff_k | |
| K2eff(k) | Vector with K2eff_k | |

TABLE 2

| | | Typ. range |
|---|---|---|
| Constant | Designation | of values |
| T1oop_{—} | Delay time (Z1 or Z2) determined | 0-5 s |
| delay | randomly by the program | |
| T1oop_{—} | Delay (S) for increment/decrement | 0.1-2 s |
| | loop | |
| Tuser_{—} | User's reaction time to be taken into | 0.1-2 s |
| delay | account | |
| K1limit_{—} | Minimum contrast range KB1 | 1% |
| reg | predetermined by guidelines | |
| K2limit_{—} | Minimum contrast range KB2 | 1% |
| reg | predetermined by guidelines | |

The variables are determined externally by the program flow; the constants are not values that can be altered externally via the program flow, but rather are predetermined by initialization, algorithms or program implementation.

It is furthermore possible to store the contrast values together with the digital image data and/or the findings in order to ensure quality assurance. It is thus possible to ensure in a comprehensible manner that the requirements that are made of the image reproduction apparatus and the operator and are necessary for the medical diagnosis have been complied with.

Although modifications and changes may be suggested by those skilled in the art, it is the intention of the inventor to embody within the patent warranted hereon all changes and modifications as reasonably and properly come within the scope of his contribution to the art.

I claim as my invention:

1. A method for checking an image reproduction device comprising the steps of:
displaying a displayed image, and, within said displayed image, displaying a test zone comprising a partial region of said displayed image;
assigning an image parameter to said test zone;
altering a partial region of said test zone with respect to said image parameter;
detecting an input by a user indicating an identification of a difference in said image parameter between said partial region of said test zone and a remainder of said test zone;
upon detection of said input, storing said image parameter; and
comparing said image parameter with a predetermined guide value for said image parameter to obtain a characterization of the quality of said displayed image.

2. A method as claimed in claim 1 wherein the step of comparing said image parameter with a predetermined guide value comprises obtaining a transformation of said image parameter and comparing said transforamtion with said predetermined guide value.

3. A method as claimed in claim 1 comprising employing a standardized test pattern as said test zone.

4. A method as claimed in claim 3 comprising employing an SMPTE test pattern as said standardized test pattern.

5. A method as claimed in claim 4 wherein said SMPTE test pattern includes 95% contrast elements and 5% contrast elements, and wherein the step of altering a partial region of said test zone comprises dynamically altering said 95% contrast elements and said 5% contrast elements.

6. A method as claimed in claim 1 comprising employing a plurality of test zones and assigning respective image parameters to said test zones.

7. A method as claimed in claim 6 wherein said image parameter has a problem range associated with the quality of said image, and wherein said parameter in said test zone initially is within said problem range, before altering said parameter.

8. A method as claimed in claim 1 comprising initiating said test method at an initiation time, and wherein the step of altering said image parameter comprises altering said image parameter with a time delay after said initiation.

9. A method as claimed in claim 6 comprising randomly selecting said time delay.

10. A method as claimed in claim 1 comprising employing luminance of said displayed image as said characteristic representing the quality of said displayed image.

11. A method as claimed in claim 1 wherein said test zone is a first test zone and comprising displaying a second test zone as a different partial region of said displayed image, said first test zone being dark and said second test zone being bright, and alternatingly altering respective partial regions of said first and second test zones with regard to said image parameter.

12. A method as claimed in claim 1 wherein said test zone has a surrounding region which surrounds said partial region of said test zone, and wherein said partial region of said test zone and said surrounding region have homogenous image parameters over their respective areas.

13. A method as claimed in claim 1 comprising selecting said image parameter from the group consisting of contrast, resolution and noise.

14. A method as claimed in claim 13 comprising employing a black-and-white image parameter as said image parameter.

15. A method as claimed in claim 1 comprising employing an image parameter representing contrast and comprising the additional step of, in any region of said displayed image wherein a contrast difference cannot be identified by comparing said image parameter with said predetermined guide value, no contrast values are employed for displaying said displayed image.

16. A method for operating a medical apparatus comprising the steps of:
executing a plurality of functional sequences with a medical apparatus including displaying a displayed image on a display device of said medical apparatus;
determining a parameter associated with behavior of said display device relative to an image parameter, dependent on predetermined boundary conditions; and
controlling at least one of said functional sequences dependent on said parameter.

17. A method as claimed in claim 16 comprising employing ambient illumination as said behavior of said display device and comparing said image parameter with said ambient illumination while executing at least one of said functional sequences and, if said image parameter and said ambient illumination deviate from each other beyond a predetermined tolerance range, controlling said at least one of said functional sequences dependent on said parameter.

18. A computer program for checking an image reproduction device wherein a test zone is displayed as a partial region of a displayed image, said computer program:
assigning an image parameter to said test zone;
altering a partial region of said test zone with respect to said image parameter;
detecting an input by a user indicating an identification of a difference in said image parameter between said partial region of said test zone and a remainder of said test zone;
upon detection of said input, storing said image parameter; and
comparing said image parameter with a predetermined guide value for said image parameter to obtain a characterization of the quality of said displayed image.
